(19) United States
(12) Patent Application Publication
(10) Pub. No.: US 2026/0283985 A1
(43) Pub. Date: Sep. 24, 2026

(54) ALDOXIME ANTIBIOTICS AND RELATED METHODS

(71) Applicant: Marshall University Research Corporation, Huntington, WV (US)

(72) Inventors: Leslie Frost, Huntington, WV (US); Lydia Bogomolnaya, Huntington, WV (US); Yevgeniy Shakirov, Huntington, WV (US)

(21) Appl. No.: 19/576,575

(22) Filed: Mar. 24, 2026

Related U.S. Application Data
(60) Provisional application No. 63/776,667, filed on Mar. 24, 2025.

(51) Int. Cl. A61K 31/15; A61K 31/404; A61K 31/4164; A61K 31/7048; A61K 38/12; A61K 38/14; C07C 251/40; C07D 209/14; C07D 233/64

(52) U.S. Cl. CPC A61K 31/15; A61K 31/404; A61K 31/4164; A61K 31/7048; A61K 38/12; A61K 38/14; C07C 251/40; C07D 209/14; C07D 233/64

(57) ABSTRACT

A compound which includes an aldoxime molecule having antibiotic properties and that is derived from an amino acid. The compound includes two substituent groups with one substituent group selected from a phenyl group, an indole group, or an imidazole group, and another substituent group selected from a hydrogen atom or a lower alkyl having 1 to 8 carbon atoms. Methods of synthesizing an aldoxime compound comprise steps of converting an amino acid to an aldehyde compound, and reacting the aldehyde compound with an amine to produce the aldoxime compound. Methods of treating a bacterial infection are further provided and comprise administering an effective amount of an aldoxime compound to a subject in need thereof.

### RELATED APPLICATIONS

This application claims priority from U.S. Provisional Application Ser. No. 63/776,667, filed Mar. 24, 2025, the entire disclosure of which is incorporated herein by this reference.

### TECHNICAL FIELD

The presently-disclosed subject matter generally relates to aldoxime compounds and related methods. In particular, certain embodiments of the presently-disclosed subject matter relate to aldoxime compounds, methods of synthesizing the aldoxime compounds from amino acids, and methods of treating a bacterial infection that make use of the aldoxime compounds as antibiotics.

### BACKGROUND

An aldoxime is an organic compound with the general formula R₁R₂C═N—OH, where R₁ is an organic sidechain and R₂ is a hydrogen. Aldoximes can exist in two isomeric forms, E or Z conformation, due to the C═N double bond, and it is appreciated that plants can synthesize aldoximes from amino acid precursor molecules (Val, Leu, Ile, Met, Trp, Phe, and Tyr) using various enzymes. These aldoximes play an important role in plant defense against herbivores and pathogens. To date, however, there is no simple method for the chemical synthesis of aldoximes from amino acids. Accordingly, a simple, economical, and green method of synthesizing aldoximes from amino acids, and in a manner that allows the aldoximes to be subsequently utilized as antibiotics for the treatment of various bacterial infections, would be both highly desirable and beneficial.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of such features.

The presently-disclosed subject matter includes aldoxime compounds, methods of synthesizing the aldoxime compounds from amino acids, and methods of treating a bacterial infection that make use of the aldoxime compounds as antibiotics. In some embodiments, a compound is provided that comprises an aldoxime having the general formula (I):

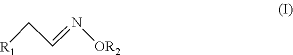

In some embodiments, in general formula (I), R₁ is selected from a phenyl group, an indole group, or an imidazole group, each of which can be optionally substituted or unsubstituted. In some embodiments, R₂ is a hydrogen atom or a lower alkyl having 1 to 8 carbon atoms, such as, in certain embodiments, a methyl group. In some embodiments, the compound of formula (I) is derived from an amino acid. In certain embodiments, the compound of general formula (I) is included in a pharmaceutical composition that further comprises a pharmaceutically-acceptable vehicle, carrier, or excipient.

In some embodiments of the presently-disclosed compounds, the compound has the following formula (II):

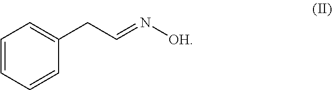

In some embodiments of the presently-disclosed compounds, the compound has the following formula (IIB):

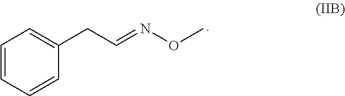

In some embodiments of the presently-disclosed compounds, the compound has the following formula (III):

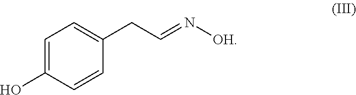 [SMILES: O/N=C/Cc1ccc(O)cc1]

In some embodiments of the presently-disclosed compounds, the compound has the following formula (IV):

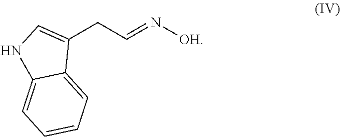 [SMILES: O/N=C/Cc1c[nH]c2ccccc12]

In some embodiments of the presently-disclosed compounds, the compound has the following formula (V):

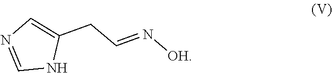

Further provided, in some embodiments of the presently-disclosed subject matter, are methods of synthesizing an aldoxime compound. In some embodiments, a method of synthesizing an aldoxime compound comprises and initial step of converting an amino acid to an aldehyde compound, and then reacting the aldehyde compound with an amine to produce the aldoxime compound. In some implementations, converting the amino acid to the aldehyde compound comprises hypochlorite-mediated decarboxylation. In some implementations, reacting the aldehyde compound with an amine comprises reacting the aldehyde compound with hydroxylamine. In some implementations of the synthesis methods, the amino acid is phenylalanine, tyrosine, tryptophan, histidine, or combinations thereof. Advantageously, in certain implementations, the steps of converting the amino acid and reacting the aldehyde compound are carried out in a single vessel.

Still further provided, in some embodiments of the presently-disclosed subject matter, are methods of treating a bacterial infection. In some implementations, a method of treating a bacterial infection comprises administering an effective amount of an aldoxime compound of general formula (I) to a subject in need thereof. In some implementations of the methods, the aldoxime compound is an aldoxime compound of formula (II), formula (IIB), formula (III), formula (IV), or formula (V), as described above. In some implementations of the therapeutic methods, an effective amount of an additional antibiotic is administered, such as, in certain implementations, carbomycin, vancomycin, or polymyxin B. In some implementations, the bacterial infection comprises a Gram-positive bacterial infection, such as, in certain implementations, a Staphylococcus aureus infection.

Further features and advantages of the present invention will become evident to those of ordinary skill in the art after a study of the description, figures, and non-limiting examples in this document.

### BRIEF DESCRIPTION OF THE DRAWINGS

FIG. 1 is a schematic diagram showing the structure of Z and E isomers of oxime molecules;

FIG. 2 is a schematic diagram showing a reaction scheme for the synthesis of phenylacetaldoxime from the amino acid phenylalanine;

FIG. 3 is a gas chromatogram of an ethyl acetate purified reaction product;

FIG. 4 is an electron impact (EI) mass spectrum of the molecule eluting at 9.60 minutes (peak 2) in FIG. 3;

FIG. 5 is an EI mass spectrum of the molecule eluting at 9.78 minutes (peak 3) in FIG. 3;

FIG. 6 is an EI mass spectrum of the molecule eluting at 8.62 minutes (peak 1) in FIG. 3;

FIG. 7 includes the chemical structure of 4-hydroxyphenylacetaldoxime;

FIG. 8 includes the chemical structure of indole-3-acetaldoxime;

FIG. 9 is a gas chromatogram of an ethyl acetate purified oxime reaction product from tyrosine precursor amino acid;

FIG. 10 includes graphs showing growth of S. aureus American Type Culture Collection (ATCC) strain 25923 in the presence of the as-synthesized aldoximes;

FIG. 11 is a line graph showing growth of MCF10A breast epithelial cells in the presence of increasing concentrations of the as-synthesized aldoximes (Phe-Oxime (formula (II)), Tyr-Oxime (formula (III)), and Trp-Oxime (Formula (IV)));

FIG. 12 is a gas chromatogram of a purified reaction product produced from an exemplary synthesis method of the presently-disclosed subject matter, utilizing phenylalanine as a precursor amino acid and methoxyamine;

FIG. 13 is an EI mass spectrum of the molecule eluting at 8.9 minutes in FIG. 12 and confirming the synthesis of O-methylated phenylacetaldoxime;

FIG. 14 is a graph showing growth of S. aureus ATCC 25923 in the presence of O-methylated phenylacetaldoxime;

FIG. 15 is a schematic diagram showing a reaction scheme for the synthesis of 2-(1H-imidazol-5-yl)acetaldoxime from the amino acid histidine; and

FIG. 16 includes bar graphs showing cytotoxicity of aldoximes toward non-tumorigenic (MCF 10A) and triple negative breast cancer (MDA-231) cells.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

The present application can “comprise” (open ended), “consist of” (closed ended), or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently-disclosed subject matter includes aldoxime compounds and related methods. In particular, certain embodiments of the presently-disclosed subject matter include aldoxime compounds, methods of synthesizing the aldoxime compounds from amino acids, and methods of treating a bacterial infection that make use of the aldoxime compounds as antibiotics.

In some embodiments of the presently-disclosed subject matter, a compound having antibacterial properties is provided that comprises an aldoxime compound derived from an amino acid. In some embodiments, a compound is provided comprising an aldoxime having the general formula (I):

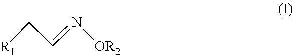

In some embodiments, in general formula (I), R₁ is selected from a phenyl group, an indole group, or an imidazole group, each of which can be optionally substituted or unsubstituted. In some embodiments, R₂ is a hydrogen atom or a lower alkyl having 1 to 8 carbon atoms, such as, in certain embodiments, a methyl group. In some embodiments, the compound of formula (I) is derived from an amino acid, including, in some embodiments, phenylalanine, tyrosine, tryptophan, histidine, or combinations thereof.

In one exemplary embodiment of the present invention, R₁ is an unsubstituted phenyl group or, in other words, a cyclic, aromatic substituent with the chemical formula C₆H₅—. In some such embodiments, the aldoxime compound is derived from the amino acid phenylalanine, R₁ is an unsubstituted phenyl group, and R₂ is a hydrogen atom, such that the aldoxime compound has the following formula (II):

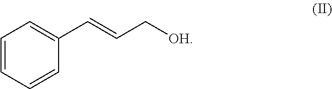

In another exemplary embodiment, R₁ is an unsubstituted phenyl group and R₂ is a lower alkyl group. The term “alkyl” is used herein to refer to C1-20 inclusive, linear (i.e., “straight-chain”), branched, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, methylpropynyl, butynyl, pentynyl, hexynyl, heptynyl, and allenyl groups. “Branched” when used herein in reference to an alkyl group refers to an alkyl group in which typically a lower alkyl group, such as methyl, ethyl or propyl, is attached to a linear alkyl chain. “Lower alkyl” refers to an alkyl group having 1 to about 8 carbon atoms (i.e., a C1-8 alkyl), e.g., 1, 2, 3, 4, 5, 6, 7, or 8 carbon atoms. “Higher alkyl” refers, on the other hand, to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C1-8 straight-chain alkyls. In other embodiments, “alkyl” refers, in particular, to C1-8 branched-chain alkyls. In some embodiments, where the aldoxime compound is derived from the amino acid phenylalanine, R₁ is an unsubstituted phenyl group and R₂ is a lower alkyl in the form of a methyl group such that the aldoxime compound has the following Formula (IIB):

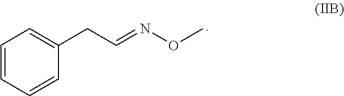

In some embodiments, the aldoxime compound is derived from the amino acid tyrosine, R₁ is an phenyl group substituted with a hydroxyl (—OH) group, and R₂ is a hydrogen atom, such that the aldoxime compound has the following Formula (III):

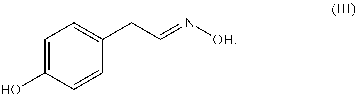 [SMILES: O/N=C/Cc1ccc(O)cc1]

In some other embodiments of the presently-disclosed subject matter, as indicated above and rather than including a phenyl group, R₁ is an indole group comprising a bicyclic aromatic heterocyclic compound including a benzene ring fused to a nitrogen-containing pyrrole ring. For instance, in some embodiments where R₁ is an indole group, the aldoxime compound is derived from tryptophan and R₂ is a hydrogen atom such that the compound has the following Formula (IV):

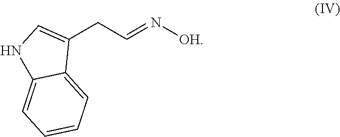 [SMILES: O/N=C/Cc1c[nH]c2ccccc12]

In other embodiments of the presently-described aldoxime compounds, R₁ comprises an imidazole group, where R₁ comprises an aromatic heterocyclic compound including a five-membered ring with three carbon atoms and two nitrogen atoms, and where the nitrogen atoms are in a 1-3 position relative to each other. In some embodiments, such an imidazole group can be substituted at any one or more of the atoms at the 1, 2, 4, and/or 5 positions. In some embodiments of the presently-disclosed compounds, the aldoxime compound is derived from histidine, R₁ comprises an unsubstituted imidazole group, and R₂ is hydrogen such that the aldoxime compound has the following formula (V):

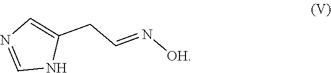

Further provided, in some embodiments, are pharmaceutical compositions comprising an aldoxime compound of the presently-disclosed subject matter, in combination with a pharmaceutically-acceptable vehicle, carrier, or excipient. Indeed, when referring to certain embodiments herein, the terms “compound” and/or “composition” can be used interchangeably herein to refer to a pharmaceutical composition that includes the exemplary aldoxime compounds.

The term “pharmaceutically-acceptable carrier” as used herein refers to sterile aqueous or nonaqueous solutions, dispersions, suspensions, or emulsions, as well as sterile powders for reconstitution into sterile injectable solutions or dispersions just prior to use. Proper fluidity can be maintained, for example, by the use of coating materials such as lecithin, by the maintenance of the required particle size in the case of dispersions and by the use of surfactants. These compositions can also contain adjuvants, such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like.

Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of compound to biodegradable polymer and the nature of the particular biodegradable polymer employed, the rate of compound release can be controlled. Depot injectable formulations can also be prepared by entrapping the compound in liposomes or microemulsions, which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

Suitable formulations can further include aqueous and non-aqueous sterile injection solutions that can contain antioxidants, buffers, bacteriostats, bactericidal antibiotics, and solutes that render the formulation isotonic with the bodily fluids of the intended recipient; and aqueous and non-aqueous sterile suspensions, which can include suspending agents and thickening agents.

The compositions can also take forms such as suspensions, solutions, or emulsions in oily or aqueous vehicles, and can contain formulatory agents such as suspending, stabilizing and/or dispersing agents. Alternatively, the compounds can be in powder form for constitution with a suitable vehicle, e.g., sterile pyrogen-free water, before use.

The formulations can be presented in unit-dose or multi-dose containers, for example sealed ampoules and vials, and can be stored in a frozen or freeze-dried (lyophilized) condition requiring only the addition of sterile liquid carrier immediately prior to use.

For oral administration, the compositions can take the form of, for example, tablets or capsules prepared by a conventional technique with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycollate); or wetting agents (e.g., sodium lauryl sulphate). The tablets can be coated by methods known in the art.

Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically-acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration, the compositions can take the form of tablets or lozenges formulated in a conventional manner.

The compositions can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt). The compounds can also be formulated in rectal compositions, creams or lotions, or transdermal patches.

Still further provided, in some embodiments of the presently-disclosed subject matter, are methods for synthesizing an aldoxime compound. In some embodiments, and as indicated above, the aldoxime compound is synthesized from an amino acid such as, in certain embodiments, phenylalanine, tyrosine, tryptophan, histidine, or combinations thereof. For instance, in one exemplary embodiment, a method of synthesizing an aldoxime molecule comprises an initial step of converting an amino acid to an aldehyde compound via a decarboxylation reaction in which an oxidant reacts with the amino group of the amino acid to cause release of the carboxyl group in the form of carbon dioxide. A number of oxidants can be utilized in this regard, including, but not limited to, sodium hypochlorite (NaOCl), hypochlorous acid (HOCl), calcium hypochlorite (Ca(OCl₂)), hydrogen peroxide (H₂O₂), sodium chlorite (NaOCl₂), and sodium chlorate (NaClO₃). In some embodiments, the oxidant is sodium hypochlorite (NaOCl) such that the amino acid undergoes hypochlorite-mediated decarboxylation to produce the aldehyde compound.

Subsequent to producing the aldehyde compound, the intermediate aldehyde compound is made to undergo a nucleophilic addition-elimination reaction in which the carbonyl group of the aldehyde is protonated to provide a suitable leaving group that, upon leaving, allows the further formation of a carbon-nitrogen double bond. In this regard, in some embodiments of the presently-described subject matter, the conversion of the intermediate aldehyde compound to the aldoxime includes a condensation reaction using an amine molecule to produce the aldoxime molecule. In some embodiments, reacting the aldehyde compound with an amine comprises reacting the aldehyde compound with a hydroxylamine (NH₂OH). Advantageously, in certain implementations, the steps of converting the amino acid and reacting the aldehyde compound are carried out in a single vessel.

In yet additional embodiments of the presently-disclosed subject matter are methods of treating a bacterial infection. In some embodiments, a method of treating a bacterial infection is provided that comprises administering an effective amount of a compound of the presently-disclosed subject matter, i.e., an aldoxime derived from an amino acid, to a subject in need thereof (i.e., a subject in need of treatment for a bacterial infection). The term “bacterial infection,” as used herein, is used to refer to any infection caused or exacerbated by the invasion, growth, and/or proliferation of bacteria on or within the body of a subject. Such infections may be present in the respiratory tract, skin, urinary tract, gastrointestinal system, blood, or ear, nose and throat of a subject, although additional sites of infection in a subject are possible as well as would be recognized by those skilled in the art. The bacterial infections can further be systemic, as may be the case with sepsis, or can be localized, such as what can occur with sinusitis. Additionally, the bacterial infections can be acute infections in which the onset of the bacterial infection is sudden, or the bacterial infection can be chronic or persistent whereby the pathogens evade the immune system and/or are resistant to common treatments.

As used herein, the terms “treatment” or “treating” thus relates to any treatment of a bacterial infection, including, but not limited to, prophylactic treatment and therapeutic treatment. As such, the terms “treatment” or “treating” include, but are not limited to: reducing the severity of a bacterial infection; inhibiting the progression of a bacterial infection; arresting or reducing the further development of a bacterial infection; ameliorating or relieving symptoms associated with a bacterial infection; and causing a regression of a bacterial infection or one or more of the symptoms associated with a bacterial infection. In some embodiments of the therapeutic methods, the subject has a bacterial infection. In some embodiments, the subject has an antibiotic resistant bacterial infection, in which the bacterial causing the infection is resistant to treatment with one or more types of antibiotics.

For administration of a therapeutic composition as disclosed herein (e.g., an aldoxime compound), conventional methods of extrapolating human dosage based on doses administered to a murine animal model can be carried out using the conversion factor for converting the mouse dosage to human dosage: Dose Human per kg=Dose Mouse per kg/12 (Freireich, et al., (1966) Cancer Chemother Rep. 50: 219-244). Drug doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich, et al. (Freireich et al., (1966) Cancer Chemother Rep. 50:219-244). Briefly, to express a mg/kg dose in any given species as the equivalent mg/sq m dose, multiply the dose by the appropriate km factor. In an adult human, 100 mg/kg is equivalent to 100 mg/kg×37 kg/sq m=3700 mg/m2.

Suitable methods for administering a therapeutic composition in accordance with the methods of the presently-disclosed subject matter include, but are not limited to, systemic administration, parenteral administration (including intravascular, intramuscular, and/or intraarterial administration), oral delivery, buccal delivery, rectal delivery, subcutaneous administration, intraperitoneal administration, inhalation, intratracheal installation, surgical implantation, transdermal delivery, local injection, intranasal delivery, and hyper-velocity injection/bombardment. Where applicable, continuous infusion can enhance drug accumulation at a target site (see, e.g., U.S. Pat. No. 6,180,082). In some embodiments, the administration of the composition is via oral administration, transdermal administration, administration by inhalation, nasal administration, topical administration, intraaural administration, rectal administration, intravenous administration, intramuscular administration, subcutaneous administration, intravitreous administration, subconjunctival administration, intracameral administration, intraocular administration or combinations thereof.

Regardless of the route of administration, the compositions of the presently-disclosed subject matter are typically administered in amount effective to achieve the desired response. As such, the term “effective amount” is used herein to refer to an amount of the therapeutic composition (e.g., an aldoxime compound and a pharmaceutically vehicle, carrier, or excipient) sufficient to produce a measurable biological response (e.g., a reduction in bacterial infection). Actual dosage levels of active ingredients in a therapeutic composition of the present invention can be varied so as to administer an amount of the active compound(s) that is effective to achieve the desired therapeutic response for a particular subject and/or application. Of course, the effective amount in any particular case will depend upon a variety of factors including the activity of the therapeutic composition, formulation, the route of administration, combination with other drugs or treatments, severity of the condition being treated, and the physical condition and prior medical history of the subject being treated. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

With respect to bacterial infections capable of being treated in accordance with the presently-disclosed subject matter, in some embodiments, the bacterial infection comprises an infection by a Gram-positive bacteria (e.g., Staphylococcus aureus, Streptococcus pyogenes, Streptococcus mutans, Enterococcus faecium) and/or an infection by a Gram-negative bacteria (e.g., Escherichia coli, Serratia marcescens, Enterobacter cloacae, Pseudomonas aeruginosa). In some embodiments, the bacterial infection is a Gram-positive bacterial infection. In some embodiments, the bacterial infection comprises an infection by a Staphylococcus aureus (e.g., methicillin resistant S. aureus (MRSA)) bacteria.

In some embodiments, the aldoxime compound of the presently-disclosed subject matter that is administered to treat a bacterial infection in a subject can be administered alone or with other additional antibiotics to treat the bacterial infection in the subject. In some embodiments, administering of the aldoxime compounds described herein with other antibiotics provides a synergistic effect where the observed therapeutic effect is greater than that observed when the antibiotics are administered alone and/or is greater than the therapeutic effects that would be observed when the individual therapeutic effects of those antibiotics are simply added together. For instance, in certain embodiments, an exemplary aldoxime compound is administered in conjunction with an effective amount of vancomycin or carbomycin to the subject as doing so leads to an enhanced treatment of the bacterial infection in the subject, such as an MRSA infection. As another example, in some embodiments, an exemplary aldoxime compound is administered in conjunction with an effective amount of polymyxin B to the subject as doing so leads to an enhanced treatment of a Gram-negative bacterial infection in the subject, such as a Pseudomonas aeruginosa infection.

For additional guidance regarding formulation and dose, see U.S. Pat. Nos. 5,326,902; 5,234,933; PCT International Publication No. WO 93/25521; Berkow et al., (1997) The Merck Manual of Medical Information, Home ed. Merck Research Laboratories, Whitehouse Station, New Jersey; Goodman et al., (1996) Goodman & Gilman's the Pharmacological Basis of Therapeutics, 9th ed. McGraw-Hill Health Professions Division, New York; Ebadi, (1998) CRC Desk Reference of Clinical Pharmacology. CRC Press, Boca Raton, Florida; Katzung, (2001) Basic & Clinical Pharmacology, 8th ed. Lange Medical Books/McGraw-Hill Medical Pub. Division, New York; Remington et al., (1975) Remington's Pharmaceutical Sciences, 15th ed. Mack Pub. Co., Easton, Pennsylvania; and Speight et al., (1997) Avery's Drug Treatment: A Guide to the Properties, Choice, Therapeutic Use and Economic Value of Drugs in Disease Management, 4th ed. Adis International, Auckland/Philadelphia; Duch et al., (1998) Toxicol. Lett. 100-101:255-263.

The presently-disclosed subject matter is further illustrated by the following specific but non-limiting examples.

### EXAMPLES

### Example 1—Aldoxime Synthesis from Amino Acids

Experiments were undertaken to develop a simple, economical, green method of aldoxime synthesis from amino acids and to then test these aldoximes for antibiotic properties.

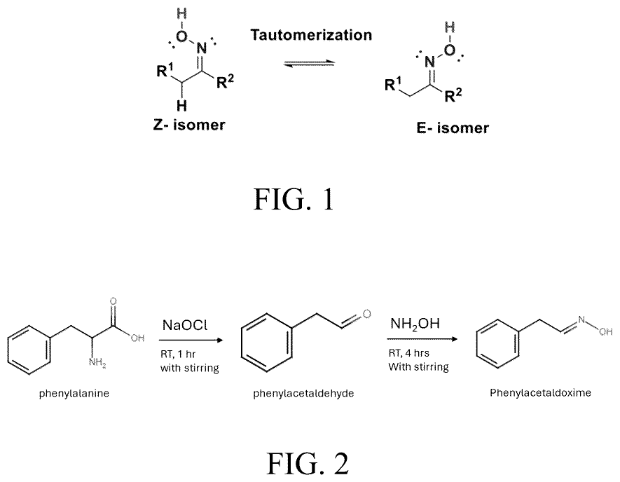

For the synthesis of the aldoxime compounds, amino acids were first converted to aldehydes via hypochlorite-mediated decarboxylation. This reaction involved adding bleach (contains hypochlorite, NaOCl) to the amino acid to produce the aldehyde compound. Next, the aldehyde was reacted with hydroxylamine to produce the aldoxime compounds. The synthesis of phenylacetaldoxime from the amino acid phenylalanine was the initial focus of the experiments to analyze reaction conditions. In general, 35 mg of phenylalanine was added to 75 ml of methanol in a 250 ml flask with stirring until the phenylalanine dissolved. Next, 400 μl of 5% NaOCl solution (active ingredient in bleach) was slowly added using a syringe pump at a rate of 20 μL/min and allowed to react at room temperature for a total of one hour. Then, 14 mg of hydroxylamine (NH₂OH) was added to the mixture and allowed to react at room temperature for at least 4 hours with stirring in a chemical fume hood. The reaction protocol is shown in FIG. 2.

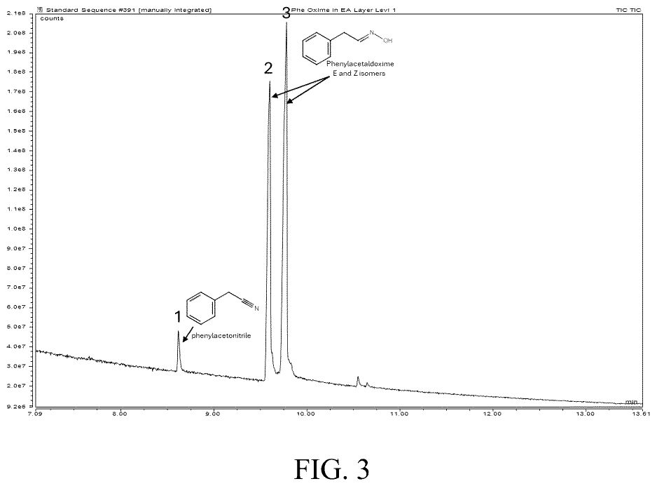

The experimental method was able to achieve a one-pot synthesis of phenylacetaldoxime at room temperature. The resulting phenylacetaldoxime was purified by evaporating off the methanol solvent in a fume hood, adding 10 ml of water and 10 ml of ethyl acetate to the remaining solid, and removing the ethyl acetate layer which contained the purified phenylacetaldehyde oxime compound. A portion of the ethyl acetate layer was diluted with additional ethyl acetate and used for GCMS analysis of the product. The gas chromatogram is shown in FIG. 3.

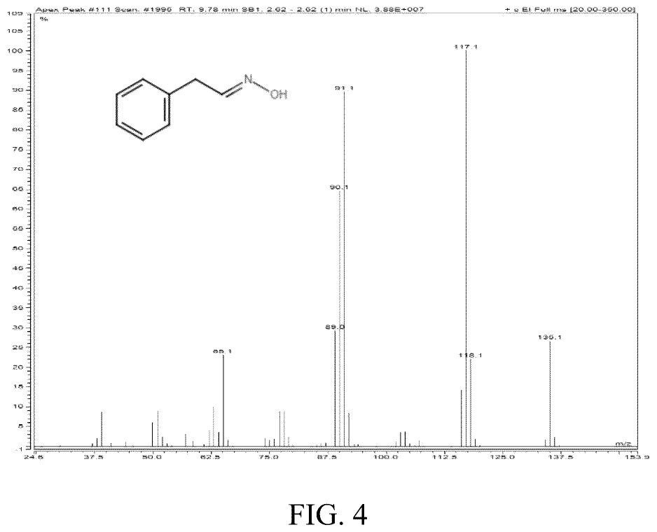

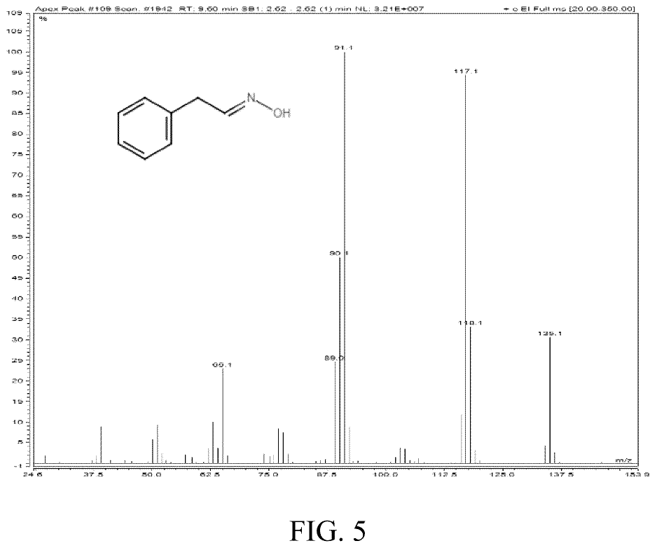

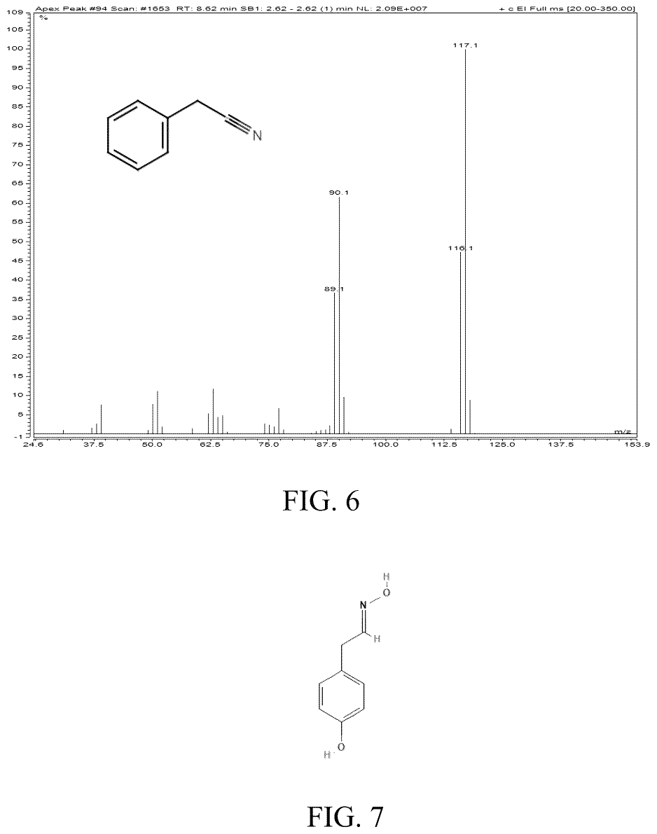

The electron impact (EI) mass spectrum of each molecule in the gas chromatogram could be used to identify the molecule. The EI mass spectrum of the molecule eluting at 9.60 minutes (peak 2 in FIG. 3) is shown in FIG. 4. The molecular weight of the molecule was 135 Da which corresponds to the molecular weight of phenylacetaldoxime. The fragments ions at 117 Da and 91 Da are the expected fragment ions for phenylacetaldoxime. The EI mass spectrum of the molecule eluting at 9.78 minutes (peak 3 in FIG. 3) is shown in FIG. 5. The spectrum matches that of peak 2 indicating that these molecules are isomers of each other. Aldoximes exist in two isomeric forms (FIG. 1), so peaks 2 and 3 are the E and Z isomeric forms of phenylacetaldoxime. The EI mass spectrum of the molecule eluting at 8.62 minutes (peak 1 in FIG. 3) is shown in FIG. 6. The fragments ions indicate that this molecule is phenylacetonitrile. This molecule was most likely created when the sample was heated to 250° C. in the injector of the GCMS instrument. Phenylacetaldoxime can lose a water molecule at high temperatures to form the nitrile version of the molecule. Samples were further purified by gradient reverse-phase HPLC using a C18 column and acetonitrile as the eluant.

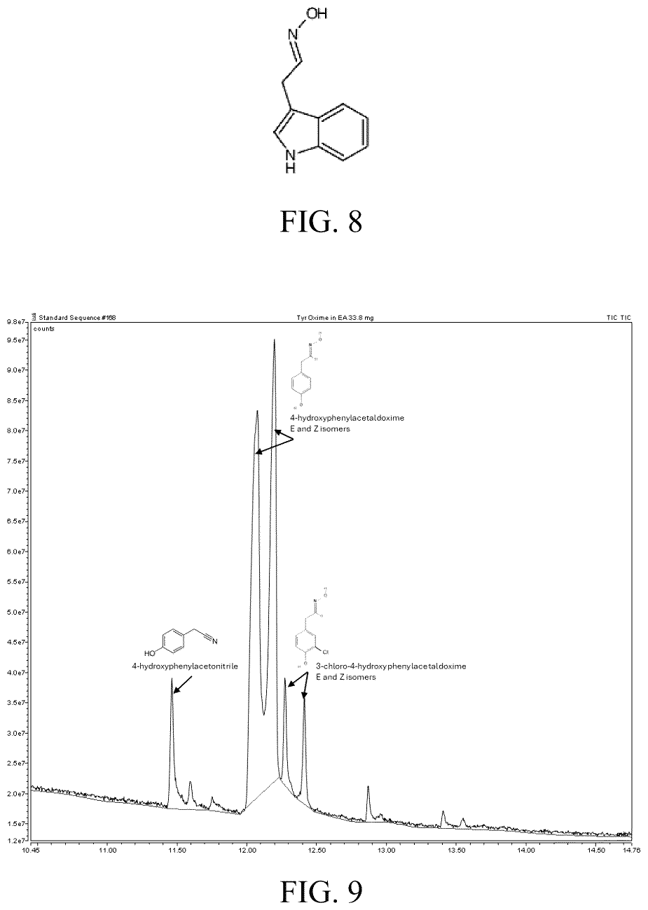

The above-described procedure was then used to synthesize an aldoxime from the amino acid tyrosine producing 4-hydroxyphenylacetaldoxime (FIG. 7) and the amino acid tryptophan producing indole-3-acetaldoxime (FIG. 8). The aldoximes produced were analyzed by GCMS. The gas chromatogram for 4-hydroxyphenylacetaldoxime is shown in FIG. 9. The electron impact (EI) mass spectrum of each molecule in the gas chromatogram was used to identify the molecule. The products from the tyrosine reaction were not as pure as those from phenylalanine, and chlorine was also added to the phenyl ring during the reaction, which was later believed to abrogate the antibacterial properties of the aldoxime compound produced from tyrosine.

The purified aldoximes were next evaluated for antibacterial properties. Each compound was reconstituted in ethyl acetate and tested against S. aureus ATCC 25923 in the range of concentrations between 0.3125-20 mg/ml. All three aldoximes were able to completely inhibit S. aureus growth (FIG. 10) with phenylacetaldoxime (Phe-Oxime, Formula II) and 4-hydroxyphenylacetaldoxime (Tyr-Oxime, Formula III) having a minimally inhibitory concentration (MIC) of 1.25 mg/ml and indole-3-acetaldoxime (Trp-Oxime, formula IV) having a higher MIC of 5 mg/ml.

### Example 2—Preparation of O-Methylated Aldoxime Analogs

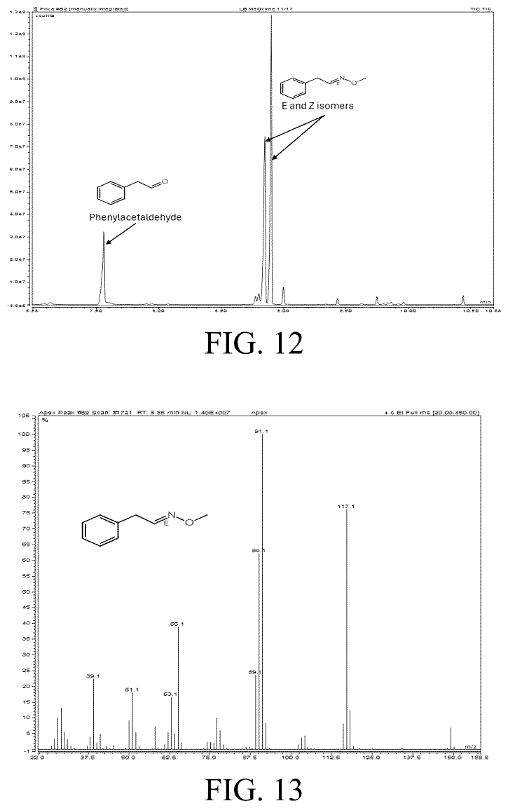

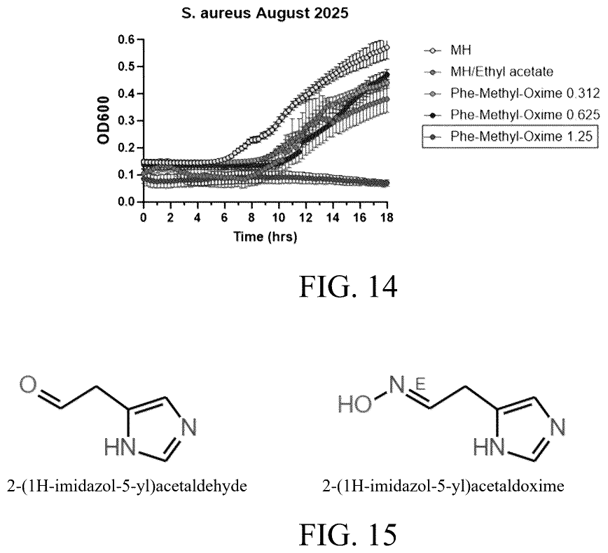

O-methylated aldoxime analogs were prepared using methoxyamine hydrochloride in place of hydroxylamine hydrochloride. Samples were synthesized by the same procedure from precursor amino acids, but methoxyamine was used to convert the aldehydes to O-methylated aldoximes with gas chromatography subsequently being utilized to confirm the chemical structures of the O-methylated phenylacetaldoxime (FIGS. 12-13). The O-methylated aldoximes were found to have comparable or better antibiotic activity against S. aureus ATCC 25923 (FIG. 14) using the procedures as described above with the initial three aldoximes that were synthesized.

### Example 3—Evaluation of Cytotoxicity of Aldoximes Toward Non-Tumorigenic (MCF 10A) and Triple Negative Breast Cancer (MDA-231) Cells

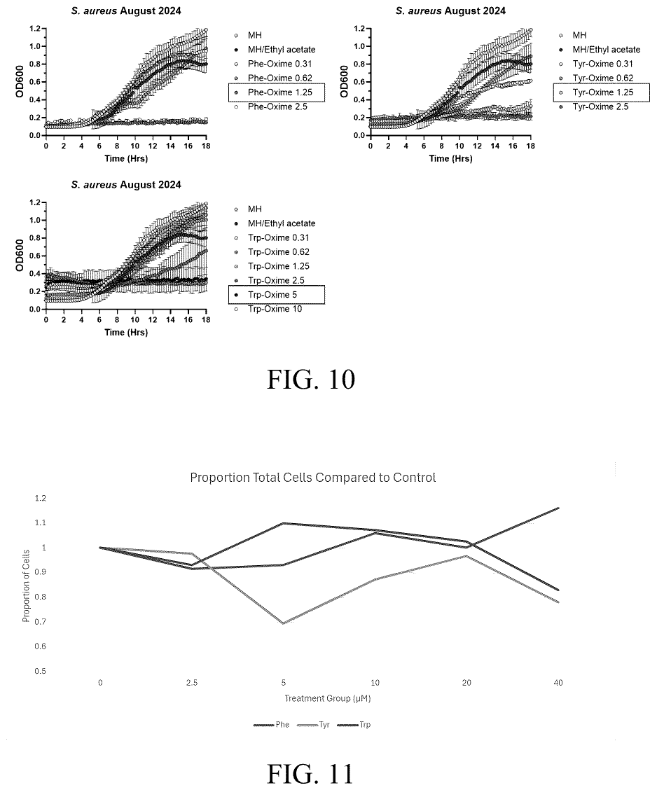

The aldoximes were then tested for mammalian cytotoxicity against MCF10A breast epithelial cells. The compounds showed no statistical cytotoxicity after 2 days of treatment (FIG. 11) even at the higher concentrations (40 μM).

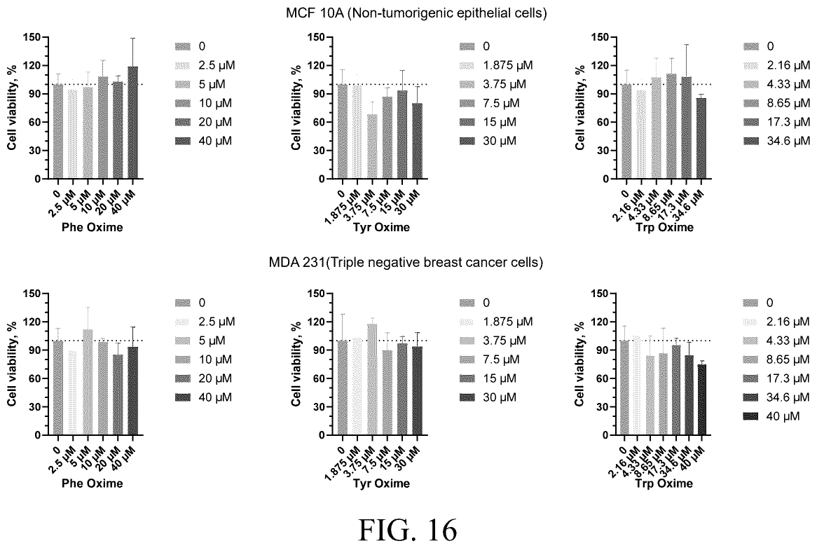

To further evaluate the as-synthesized compounds, both MCF10A and MDA-231 cells were grown in DMEM F12 cell culture medium to confluency. The synthesized aldoximes were then reconstituted in DMSO and added to cells in amounts ranging from 0 to 40 μM. The cells were subsequently incubated for 2 days in 5% CO₂ at 37° C., at which time cell viability was assessed by analyzing Trypan Blue-stained cells using Coutess II Automated Cell Counter (ThermoFisher). The experiments were done in triplicate, and the data was analyzed using one-way ANOVA. As shown in FIG. 16, Trp-Oxime (Formula IV), Tyr-Oxime (Formula III) and Phe-Oxime (Formula II) did not have any cytotoxicity toward M10A (normal breast epithelial cells) and MDA231 triple negative breast cancer cells.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Maresh, J., Crowe, S., Ralko, A., Aparece, M., Murphy, C., Krzeszowiec, M., and Mullowney, M. Tetrahedron Letters 55, 5047-5051 (2014).

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A compound, comprising an aldoxime molecule having the general formula (I): 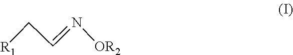
wherein R₁ is selected from a phenyl group, an indole group, or an imidazole group, each of which optionally substituted or unsubstituted,
wherein R₂ is a hydrogen atom or a lower alkyl having 1 to 8 carbon atoms, and
wherein the compound of formula (I) is derived from an amino acid.

2. The compound of claim 1, wherein the compound has the following formula (II):

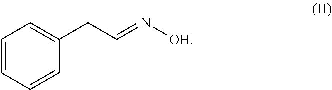

3. The compound of claim 1, wherein the compound has the following formula (IIB):

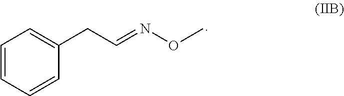

4. The compound of claim 1, wherein the compound has the following formula (III):

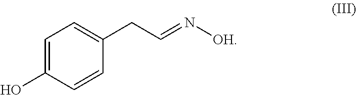 [SMILES: O/N=C/Cc1ccc(O)cc1]

5. The compound of claim 1, wherein the compound has the following formula (IV):

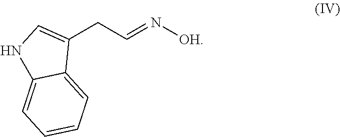 [SMILES: O/N=C/Cc1c[nH]c2ccccc12]

6. The compound of claim 1, wherein the compound has the following formula (V):

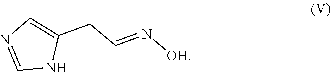

7. A pharmaceutical composition, comprising a compound of claim 1 and a pharmaceutically-acceptable vehicle, carrier, or excipient.

8. A method of synthesizing an aldoxime compound, comprising:
converting an amino acid to an aldehyde compound; and
reacting the aldehyde compound with an amine to produce the aldoxime compound.

9. The method of claim 8, wherein converting the amino acid to the aldehyde compound comprises hypochlorite-mediated decarboxylation.

10. The method of claim 8, wherein reacting the aldehyde compound with an amine comprises reacting the aldehyde compound with hydroxylamine.

11. The method of claim 8, wherein the amino acid is selected from the group consisting of phenylalanine, tyrosine, tryptophan, histidine, and combinations thereof.

12. The method of claim 8, wherein the steps of converting the amino acid and reacting the aldehyde compound are carried out in a single vessel.

13. A method of treating a bacterial infection, comprising administering an effective amount of a compound of claim 1 to a subject in need thereof.

14. The method of claim 13, wherein the compound has the following formula (II):

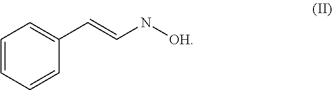

15. The method of claim 13, wherein the compound has the following formula (IIB):

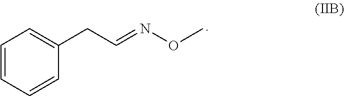

16. The method of claim 13, wherein the compound has the following formula (III):

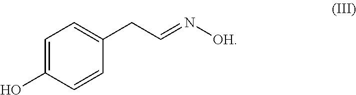 [SMILES: O/N=C/Cc1ccc(O)cc1]

17. The method of claim 13, wherein the compound has the following formula (IV):

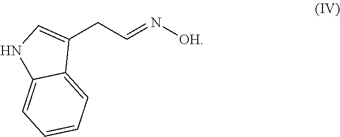 [SMILES: O/N=C/Cc1c[nH]c2ccccc12]

18. The method of claim 13, wherein the compound has the following formula (V):

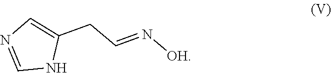

19. The method of claim 13, further comprising administering an additional antibiotic to the subject.

20. The method of claim 19, wherein the additional antibiotic comprises carbomycin, vancomycin, or polymyxin B.

21. The method of claim 13, wherein the bacterial infection comprises a gram positive bacterial infection.

22. The method of claim 21, wherein the gram positive bacterial infection is a Staphylococcus aureus infection.
